SEQUENCE LISTING (4 sequences)

SEQ ID NO: 1 - protein, 7 aa, Artificial
  Synthetic C-peptide
XXXXXXX

SEQ ID NO: 2 - protein, 7 aa, Artificial
  Synthetic C-peptide
XGXGXGX

SEQ ID NO: 3 - protein, 7 aa, Artificial
  Synthetic C-peptide
TGLGSGK

SEQ ID NO: 4 - protein, 8 aa, Artificial
  Synthetic C-peptide
GTGLGSGK

(19) United States
(12) Patent Application Publication
(10) Pub. No.: US 2009/0069216 A1
(43) Pub. Date: Mar. 12, 2009

(54) Single-Chain Insulin Analogues and Pharmaceutical Formulations Thereof

(75) Inventors: Helle Naver, Allerod (DK); Thomas Borglum Kjeldsen, Virum (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk A/S, Bagsvaerd (DK)

(21) Appl. No.: 12/280,183

(22) PCT Filed: Feb. 19, 2007
(86) PCT No.: PCT/EP07/51568

§ 371 Date: Oct. 8, 2008

(30) Foreign Application Priority Data
Feb. 21, 2006 (EP) 06110204.2

(51) Int. Cl. A61K 38/28; C07K 14/62; A61P 3/10

(52) U.S. Cl. 514/4; 530/303

(57) ABSTRACT

The present invention is related to fast acting single-chain insulin comprising a modified B-chain and the A-chain of human insulin or an analogue thereof connected by a connecting peptide wherein one or more of the amino acid residues in position B25, B26 or B27 in the human B-chain are Glu or Asp or are deleted and/or B28 is Glu, Asp, Lys or is deleted, and the amino acid residue in position B10 in the human insulin B-chain is Gln, Ala, Val, Thr or Ser. The invention is also related to pharmaceutical compositions being a mixture of the rapid acting single-chain insulin and the protracted acylated insulin.

### FIELD OF THE INVENTION

The present invention is related to fast acting, single-chain insulin analogues and pharmaceutical compositions comprising the fast acting, single-chain insulin analogues and pharmaceutical compositions comprising the single-chain insulin analogues in mixture with an acylated, long action insulin analogue.

### BACKGROUND OF THE INVENTION

Insulin is a polypeptide hormone secreted by β-cells of the pancreas and consists of two polypeptide chains, A and B, which are linked by two inter-chain disulphide bridges. Furthermore, the A-chain features one intra-chain disulphide bridge.

The hormone is synthesized as a single-chain precursor of proinsulin (preproinsulin) consisting of a prepeptide of 24 amino acid followed by proinsulin containing 86 amino acids in the configuration: prepeptide B-Arg Arg-C-Lys Arg-A, in which C is a connecting peptide of 31 amino acids. Arg-Arg and Lys-Arg are cleavage sites for cleavage of the connecting peptide from the A and B chains to form the two-chain insulin molecule. Insulin is essential in maintaining normal metabolic regulation.

The hormone is secreted as defined hexamers which dissociate by dilution firstly into dimers and secondly into monomers. The active hormone is the insulin monomer. Destabilization of the insulin hexamer and/or the insulin dimer results in fast acting insulins like B28 Asp human insulin which is disclosed in EP 214826. Another type of fast acting insulins such as LysB28ProB29 human insulin is disclosed in U.S. Pat. No. 5,474,978.

Currently, the treatment of diabetes, both type 1 diabetes and type 2 diabetes, relies to an increasing extent on the so-called intensive insulin treatment. According to this regimen, the patients are treated with multiple daily insulin injections comprising one or two daily injections of a long acting insulin to cover the basal insulin requirement supplemented by bolus injections of a fast acting insulin to cover the insulin requirement related to meals.

Long acting insulin compositions are well known in the art. Thus, one main type of long acting insulin compositions comprises injectable aqueous suspensions of insulin crystals or amorphous insulin. In these compositions, the insulin compounds utilized typically are protamine insulin, zinc insulin or protamine zinc insulin.

Certain inconveniencies are associated with the use of insulin suspensions. Thus, in order to secure an accurate dosing, the insulin particles must be suspended homogeneously by gentle shaking before a defined volume of the suspension is withdrawn from a vial or expelled from a cartridge. Also, for the storage of insulin suspensions, the temperature must be kept within more narrow limits than for insulin solutions in order to avoid lump formation or coagulation.

Another type of long acting insulin compositions are solutions having a pH value below physiological pH from which the insulin will precipitate because of the rise in the pH value when the solution is injected. A drawback with these solutions is that the particle size distribution of the precipitate formed in the tissue on injection, and thus the release profile of the medication, depends on the blood flow at the injection site and other parameters in a somewhat unpredictable manner. A further drawback is that the solid particles of the insulin may act as a local irritant causing inflammation of the tissue at the site of injection.

A further group of long acting or protracted insulin derivates are acylated insulin derivates. Human insulin has three primary amino groups: the N-terminal group of the A-chain and of the B-chain and the ε-amino group of the lysine residue in position B29. Soluble insulin derivatives containing lipophilic substituents linked to the ε-amino group of a lysine residue in any of the positions B26 to B30 are disclosed in e.g. WO 95/07931 (Novo Nordisk A/S), WO 96/00107 (Novo Nordisk A/S), WO 97/31022 (Novo Nordisk A/S) and WO 2005/012347.

The protracted action has been explained partly by reversible binding to albumin and partly by formation of larger multimers than hexamers (Markussen, Diabetologia, 39, 281-288, 1996 and Havelund et al. 2004, Pharmaceutical research, 21, 1498-1504). These insulin derivatives have a prolonged profile of action and are soluble at physiological pH values.

Because diabetic patients are treated with multiple daily injections including protracted insulin supplied with multiple injections of a fast acting insulin, a combination of fast and long acting insulin in one injection could potentially save many injections.

Mixtures of long acting and fast acting insulins are generally suspensions of insulin crystals mixed with insulin in solution. WO 97/48414 and WO 97/48413 disclose mixed suspensions of the fast acting insulin analogue B29Asp human insulin. Such mixed suspensions suffer of the same potential inconveniencies as explained above, e.g. the need to suspend the insulin particles by gentle shaking before a defined insulin dosage can be withdrawn from the vial or injected from a prefilled cartridge.

WO 2003/094956 and WO 2003/094951 disclose insulin formulations prepared by mixing the fast acting insulin B28Asp human insulin and a soluble, acylated long acting insulin analogue insulin detemir (Lys^{B29} (N^{ε}-tetradecanoyl) des(B30) human insulin).

Single-chain insulins have a much higher physical stability than the well known two-chain human insulin and two-chain human insulin analogues. WO 2005/0542941 discloses a certain group of single-chain insulins with high stability and biological insulin activity.

It is the object of the present invention to provide single-chain, fast acting insulins which have improved properties over the known compounds with respect to mixability with soluble, long acting insulin analogues and an improved physical stability.

### SUMMARY OF THE INVENTION

In one aspect the present invention is related to fast acting single-chain insulin analogues comprising a modified B-chain and the A-chain of human insulin or an analogue thereof connected by a connecting peptide wherein
a) one or more of the amino acid residues in position B25, B26 or B27 in the human B-chain are Glu or Asp or are deleted and/or B28 in the human B-chain is Glu, Asp, Lys or is deleted,
b) the amino acid residue in position B10 in the human insulin B-chain is selected from the group consisting of Gln, Ala, Val, Thr and Ser, and
c) optionally the amino acid residues in position B22 is Glu or Asp, provided that if B28 is Lys then B29 is Pro.

The connecting peptide is typically shorter than the natural C-peptide of human insulin. Thus, it is typically not longer than about 15-20 amino acid residues. Examples of useful C-peptide are such with from 6-10, 6-9, 6-8, 6-7, 7-8, 7-9, or 7-10 amino acid residues in the chain.

In one embodiment the amino acid residue in position B28 is Glu or Asp.

In a further embodiment the amino acid residue in position B10 is Gln.

In another embodiment the amino acid residue in position B22, B25, B26 and B27 are the natural amino acid at that position in the human B-chain and the amino acid residue in position B28 is Glu or Asp.

In another embodiment the amino acid residues in position B22, B25, B26 and B27 are the natural amino acid at that position in the human B-chain and the amino acid residue in position B28 is Glu or Asp and the amino acid residue in position B10 is Gln.

In a further embodiment the amino acid residues in position B22, B26 and B27 are the natural amino acid at that position in the human B-chain, the amino acid residue in position B25 is Glu or Asp or is deleted, the amino acid residue in position B28 is Glu or Asp and the amino acid residue in position B10 is Gln.

In a further embodiment the amino acid residues in position B22, B25, and B27 are the natural amino acid at that position in the human B-chain, the amino acid residue in position B26 is Glu or Asp or is deleted, the amino acid residue in position B28 is Glu or Asp and the amino acid residue in position B10 is Gln.

In a further embodiment the amino acid residues in position B22, B25 and B26 are the natural amino acid at that position in the human B-chain, the amino acid residue in position B27 is Glu or Asp or is deleted, the amino acid residue in position B28 is Glu or Asp and the amino acid residue in position B10 is Gln.

In a still further embodiment the amino acid residues in position B22, B25, B26 and

B27 are the natural amino acid at that position in the human B-chain, the amino acid residue in position B28 is Lys, the amino acid residue in position B29 is Pro and the amino acid residue in position B10 is Gln.

The A- and B-chains may be further modified to improve physical and/or chemical stability as it is well known in the art. Thus the amino acid residue in position A21 of the A chain may be substituted by any other codable amino acid residue except Cys.

In one embodiment the amino acid residue in position A21 may be selected from the group consisting of Ala, Gln, Glu, Gly, His, Ile, Leu, Met, Ser, Thr, Trp, Tyr or Val, in particular Gly, Ala, Ser, and Thr. In a further embodiment A21 is Gly.

Also B1 may be modified to e.g. Asp or Gly or may be deleted, B3 may be modified to a Gln residue and A18 may be modified to a Gln residue. Furthermore the amino acid residue in position B30 may be deleted.

In another embodiment the C-peptide is a peptide sequence with the following formula Xₐ-X_{b}X_{c}-X_{d}-Xₑ-X_{f}-X_{g} (SEQ ID NO:1) wherein

Xₐ is selected from the group consisting of L, R, T, A, H, Q, G, S and V;

X_{b} is selected from the group consisting of W, G, S, A, H, R, and T;

X_{c} is selected from the group consisting of L, Y, M, H, R, T, Q, K, V, S, A, G and P;

X_{d} is selected from the group consisting of R, A, Y, M, S, N, H, and G;

Xₑ is selected from the group consisting of S, R, A, T, K P, N M, H, Q, V, and G;

X_{f} is selected from the group consisting of G and A; and

X_{g} is selected from the group consisting of K, R, P, H, F, T, I, Q, W, and A

In a further embodiment

Xₐ is selected from the group consisting of L, R, T, A, H and V;

X_{b} is selected from the group consisting of W, G, S, A, H, R, and T;

X_{c} is selected from the group consisting of L, Y, M, H, R, T, Q, K, V, S, A, G and P;

X_{d} is selected from the group consisting of R, A, Y, M, S, N, H, and G;

Xₑ is selected from the group consisting of S, R, A, T, K P, and N;

X_{f} is G; and

X_{g} is selected from the group consisting of K, R, Q and P;

In a further embodiment

Xₐ is selected from the group consisting of T, A V, K;

X_{b} is G;

X_{c} is selected from the group consisting of L, Y, M, H, R K, W;

X_{d} is G;

Xₑ is selected from the group consisting of S, K;

X_{f} is G, and

X_{g} is selected from the group consisting of K, R, Q.

In a still further embodiment the C peptide has the sequence Y₁-G-Y₂-G-Y₃-G-Y₄ (SEQ ID NO:2)

wherein

Y₁ selected from the group consisting of Val, Leu, Arg, Thr, Ala, His, Gln, Gly or Ser, Y₂ is selected from the group consisting of Leu, Tyr, Met, His, Arg, Thr, Gln, Lys, Val, Ser, Ala, Gly, Pro,

Y₃ is selected from the group consisting of Ser, Arg, Ala, Thr, Lys, Pro, Asn, Met, His, Gln, Val, Gly, and

Y₄ is Lys or Arg.

In a still further aspect Y₁ selected from the group consisting of Val, Leu, Arg, Thr, Ala, and His,

Y₂ is selected from the group consisting of Leu, Tyr, Met, and His,

Y₃ is selected from the group consisting of Ser, Arg, Ala, Thr, Lys, Pro and Asn and

Y₄ is Lys or Arg.

In another embodiment the C-peptide has the sequences TGLGSGK (SEQ ID NO:3) or GTGLGSGK (SEQ ID NO:4).

The invention is also related to a pharmaceutical composition containing the single-chain insulin analogues of the invention and optionally one or more agents suitable for stabilization, preservation or isotonicity, for example, zinc ions, phenol, cresol, a parabene, sodium chloride, glycerol or mannitol. The zinc content of the formulations may be between 0 and about 4 zinc atoms per insulin hexamer.

In another embodiment the present invention is related to a soluble, pharmaceutical soluble formulation comprising a fast acting, single-chain human insulin analogue according to the present invention in mixture with a long acting, acylated human insulin analogue together with suitable adjuvants and additives such as one or more agents suitable for stabilization, preservation or isotoni, for example, zinc ions, phenol, cresol, a parabene, sodium chloride, glycerol or mannitol.

The zinc content may be between 0 and about 4 zinc atoms per insulin hexamer. The pH of the pharmaceutical preparation may be between about 4 and about 8.5, between about 4 and about 5 or between about 6.5 and about 7.5.

Thus, an another aspect the present invention is related to a pharmaceutical formulation comprising a fast acting, single-chain insulin comprising a modified B-chain and the A-chain of human insulin or an analogue thereof connected by a connecting peptide wherein
a) one or more of the amino acid residues in position B25, B26 or B27 in the human B-chain are Glu or Asp or are deleted and/or B28 is Glu, Asp, Lys or is deleted,
b) the amino acid residue in position B10 in the human insulin B-chain is selected from the group consisting of Gln, Ala, Val, Thr and Ser, and
c) optionally the amino acid residues in position B22 is Glu or Asp provided that if B28 is Lys then B29 is Pro,
   in mixture with a long acting, acylated human insulin analogue together with suitable adjuvants and additives.

In one embodiment the present invention is related to a pharmaceutical formulation comprising a fast acting single-chain insulin analogue according to the present invention wherein the amino acid residues in position B22, B25, B26 and B27 are the natural amino acid at that position in the human B-chain, the amino acid residue in position B28 is Glu or Asp and the amino acid residue in position B10 is Gln in mixture with a long acting, acylated human insulin analogue together with suitable adjuvants and additives.

In a further embodiment the invention is related to a pharmaceutical formulation comprising a fast acting single-chain insulin analogue according to the present invention wherein the amino acid residues in position B22, B26 and B27 are the natural amino acid at that position in the human B-chain, the amino acid residue in position B25 is Glu or Asp or is deleted, the amino acid residue in position B28 is Glu or Asp and the amino acid residue in position B10 is Gln in mixture with a long acting, acylated human insulin analogue together with suitable adjuvants and additives.

In a further embodiment the invention is related to a pharmaceutical formulation comprising a fast acting single-chain insulin analogue according to the present invention wherein the amino acid residues in position B22, B25, and B27 are the natural amino acid at that position in the human B-chain, the amino acid residue in position B26 is Glu or Asp or is deleted, the amino acid residue in position B28 is Glu or Asp and the amino acid residue in position B10 is Gln in mixture with a long acting, acylated human insulin analogue together with suitable adjuvants and additives.

In a further embodiment invention is related to a pharmaceutical formulation comprising a fast acting single-chain insulin analogue according to the present invention wherein the amino acid residues in position B22, B25 and B26 are the natural amino acid at that position in the human B-chain, the amino acid residue in position B27 is Glu or Asp or is deleted, the amino acid residue in position B28 is Glu or Asp and the amino acid residue in position B10 is Gln in mixture with a long acting, acylated human insulin analogue together with suitable adjuvants and additives.

In a still further embodiment the invention is related to a pharmaceutical formulation comprising a fast acting single-chain insulin analogue according to the present invention wherein the amino acid residues in position B22, B25, B26 and B27 are the natural amino acid at that position in the human B-chain, the amino acid residue in position B28 is Lys, the amino acid residue in position B29 is Pro and the amino acid residue in position B10 is Gln in mixture with a long acting, acylated human insulin analogue together with suitable adjuvants and additives.

In one embodiment the invention is relates to a pharmaceutical composition comprising a single-chain insulin analogue according to the invention in mixture with Lys^{B29}(N^{ε}-etetradecanoyl) des(B30) human insulin (insulin detemir).

In another embodiment the invention is relates to a pharmaceutical composition comprising a single-chain insulin analogue according to the invention in mixture with Lys^{B29}(N^{ε}-(N-lithocholyl-γ-glutamyl)) des(B30) human insulin.

In a further embodiment the invention is relates to a pharmaceutical composition comprising a single-chain insulin analogue according to the invention in mixtureN^{εB29}—(N^{α—(HOOC(CH}₂)₁₄CO)-γ-Glu) des(B30) human insulin.

Representative embodiments of pharmaceutical preparations according to the present invention are mixtures of:

B(1-29)-B10 Gln-B28Glu-TGLGSGK (SEQ ID NO:3)-A(1-21) human insulin and Lys^{B29} (N^{ε}-tetradecanoyl) des(B30) human insulin;

B(1-29)-B10Gln-B28Glu-GTGLGSGK (SEQ ID NO:4)-A(1-21) human insulin and Lys^{B29}(N^{ε}-tetradecanoyl) des(B30) human insulin;

B(1-29)-B10Gln-B28Glu-TGLGSGK (SEQ ID NO:3)-A(1-21) human insulin and Lys^{B29} (N^{ε}—(N-lithocholyl-glutamyl)) des(B30) human insulin;

B(1-29)-B10Gln-B28Glu-GTGLGSGK (SEQ ID NO:4)-A(1-21) human insulin and Lys^{B29}(N^{ε}—(N^{ε}-lithocholyl-γ-glutamyl)) des(B30) human insulin;

B(1-29)-B10Gln-B28Glu-TGLGSGK (SEQ ID NO:3)-A(1-21) human insulin and N^{εB29}—(N^{α}—(HOOC(CH₂)₁₄CO)-γ-Glu) des(B30) human insulin; and

B(1-29)-B10Gln-B28Glu-GTGLGSGK (SEQ ID NO:4)-A(1-21) human insulin and N^{εB29}—(N^{α}—(HOOC(CH₂)₁₄CO)-γ-Glu) des(B30) human insulin.

In a further aspect the present invention is related to the use of the fast acting, single-chain insulin analogues according to the present invention for the preparation of a pharmaceutical preparation for the reducing of blood glucose level in mammalians in particularly for the treatment of diabetes optionally in mixture with a protracted acylated human insulin analogue.

In a further embodiment the present invention is related to a method of reducing the blood glucose level in mammalians by administrating a therapeutically active dose of a fast acting, single-chain insulin analogue according to the invention to a patient in need of such treatment optionally in mixture with a protracted acylated human insulin analogue.

### BRIEF DESCRIPTION OF THE DRAWINGS

FIG. 1 shows gelfiltration of B(1-29)-B10Gln-B28Glu-TGLGSGK (SEQ ID NO:3)-A(1-21)-A18Gln human insulin (denoted SCI:TGLGSGK(B10QB28EA18Q in the figure) mixed with Lys^{B29}(N^{ε}-tetradecanoyl) des(B30) human insulin (insulin detemir) and RVP (reverse phase) analysis of collected fractions from the gelfiltration and reversed phase HPLC identification of insulin aggregates in gel filtration fractions

FIG. 2 shows gelfiltration of B(1-29)-B10Gln-B28Glu-TGLGSGK (SEQ ID NO:3)-A(1-21)-A18Gln human insulin (denoted SCI:TGLGSGK(B10QB28EA18Q in the figure) mixed with Lys^{B29} (N^{ε}—(N-lithocholyl-γ-glutamyl)) des(B30) human insulin and RVP analysis of collected fractions from the gelfiltration and reversed phase HPLC identification of insulin aggregates in gel filtration fractions.

### DETAILED DESCRIPTION OF THE INVENTION

The single-chain insulins according to the present invention are modified at certain positions in the insulin molecule which have an impact on the formation of dimers and hexamers and the present invention is based on the surprising recognition that such single-chain insulins modified according to the present invention remain monomeric and fast acting when mixed with the long acting acylated insulins such as Lys^{B29} (N^{ε}-tetradecanoyl) des(B30) human insulin (insulin detemir), Lys^{B29} (N^{ε}-(N-lithocholyl-y-glutamyl)) des(B30) human insulin and N^{εB29}—(N^{α}—(HOOC(CH₂)₁₄CO)-γ-Glu) des(B30) human insulin.

The single-chain insulin analogues according to the invention has a much higher stability compared to the know, two-chain fast acting insulin and have furthermore shown to be mixable with the long acting, acylated insulins without loosing it fast acting insulin effect.

The long acting, acylated insulin analogues which are mixable with the single-chain insulins according to the present invention may be acylated at various positions in the insulin molecule. In one aspect the insulin is acylated in the ε-amino group of a Lys residue in a position in the B-chain of the insulin molecule in particularly in the ε-amino group of the B29 lysine group in the human insulin molecule.

The acyl group will be a liphophilic group and will typically be a fatty acid moiety comprising from about 6 to about 32, more typically from 6 to 24, from 8 to 20, from 12 to 20, from 12-16, from 10-16, from 10-20, from 14-18 or from 14-16 carbon atoms. Examples of fatty acids are capric acid, lauric acid, tetradecanoic acid (myristic acid), pentadecanoic acid, palmitic acid, heptadecanoic acid, stearic acid, dodecanoic acid, tridecanoic acid, and tetradecanoic acid. The acyl group may also be derived from a dicarboxylic fatty acid or it may be a lithocholic acid.

The acyl group may be attached directly to the free amino group in question. However, the acyl group may also be attached via amide bonds by a linker which links the free amino group in the insulin molecule and the acyl group in question together.

The acylated insulin may have one or two additional negative net charge compared to human insulin. The additional negative charge may be provided by a free carboxylic acid group in the fatty acid or by the linker group which may comprise one or more amino acid residues of which at least one will contain a free carboxylic acid or a group which is negatively charged at neutral pH.

Non limiting examples of acylated insulin analogues are Lys^{B29} (N^{ε}-tetradecanoyl) des(B30) human insulin, Lys^{B29} (N^{ε}-hexadecanoyl) des(B30) human insulin; Lys^{B29}(N^{ε}-tetradecanoyl) human insulin; Lys^{B29} (N^{ε}-hexadecanoyl) human insulin; Lys^{B29}(N^{ε}—(N^{ε}-hexadecanoyl-Glu) des(B30) human insulin; Lys^{B29}(N^{ε}—(N-lithocholyl-γ-Glu)) des(B30) human insulin; Lys^{B229} (N^{ε}-(ω-carboxyheptadecanoyl)) des(B30) human insulin; Lys^{B29}(N^{ε}-(ω-carboxyheptadecanoyl)) human insulin, N^{εB29}—(N^{α}—(HOOC(CH₂)₁₃CO)-γ-Glu) des(B30) human insulin; N^{εB29}—(N^{α}—(HOOC(CH₂)₁₄CO)-γ-Glu) des(B30) human insulin; N^{εB29}—(N^{α}—(HOOC(CH₂)₁₅CO)-γ-Glu) des(B30) human insulin; N^{εB29}—(N^{α}—(HOOC(CH₂)₁₆CO)-γ-Glu) des(B30) human insulin; N^{εB29}—(N^{α}—(HOOC(CH₂)₁₇CO)-γ-Glu) des(B30) human insulin; N^{εB29}-(N^{α}-(HOOC(CH₂)₁₈CO)-γ-Glu) des(B30) human insulin; (N^{εB29}—(N^{α}—(HOOC(CH₂)₁₃CO)-γ-Asp) des(B30) human insulin; and N^{εB29}—(N^{α}—(HOOC(CH₂)₁₄Co)-γ-Asp) des(B30) human insulin.

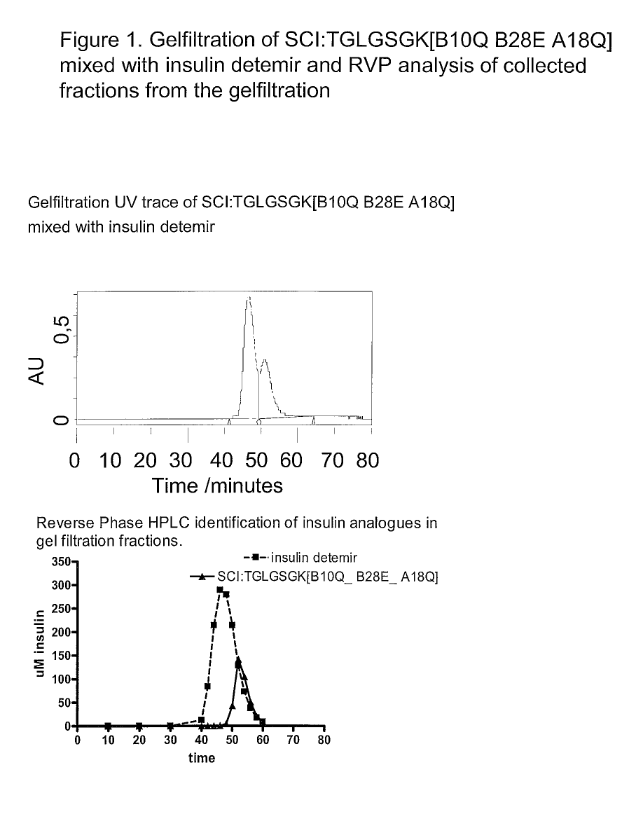

The mixability of the single-chain insulins and the acylated insulins is demonstrated in the gelfiltration test the result of which is shown in the figures. The upper curve in FIG. 1 demonstrates that the insulin detemir dodecamer migrates before the insulin SCI-monomer in gelfiltration. The uv trace demonstrates that protein with the size and amount of insulin detemir is eluted first. Protein with the expected size and concentration of the SCI:TGLGSGK[B10QB28EA18Q] monomer is eluted after the insulin detemir. This indicates that the two insulin pools do not influence each other. Since the rapid action of the monomeric insulin is dependent on the fact that this insulin is not assembled into hexamers or other aggregates, the observation that the monomeric insulin is monomeric in gelfiltration after mixing indicates rapid action in vivo too and thereby mixability with insulin detemir.

The lower curve in FIG. 1 shows a reverse phase HPLC. Fractions of the gelfiltration above was collected and the insulin content was characterized on reverse phase HPLC which separates the insulin detemir and SCI:TGLGSGK[B10Q B28E A18Q]. The uv trace which corresponds to the dodecameric insulin consists of insulin detemir and the monomeric SCI:TGLGSGK[B10Q B28E A18Q] is only found in the monomeric fraction. This confirms the indication of the uv gelfiltration trace.

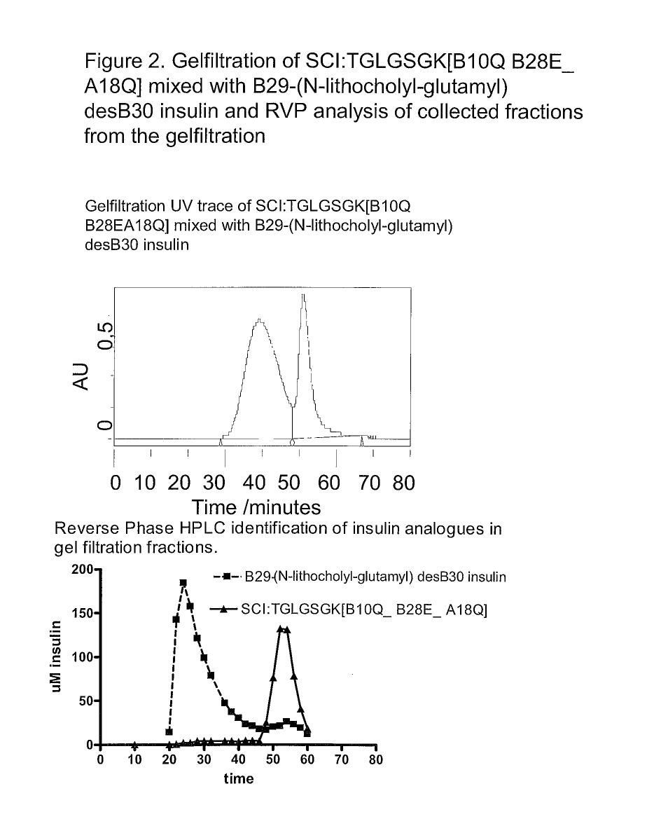

Likewise, FIG. 2 demonstrates mixability of the single-chain insulin with the acylated insulin analogue Lys^{B29} (N^{ε}—(N-lithocholyl-glutamyl)) des(B30) human insulin.

The single-chain insulins are produced by expressing a DNA sequence encoding the single-chain insulin in question in a suitable host cell by well known technique as disclosed in e.g. EP 1692168. The single-chain insulin is either expressed directly or as a precursor molecule which has an N-terminal extension on the B-chain. This N-terminal extension may have the function of increasing the yield of the directly expressed product and may be of up to 15 amino acid residues long. The N-terminal extension is to be cleaved of in vitro after isolation from the culture broth and will therefore have a cleavage site next to B1. N-terminal extensions of the type suitable in the present invention are disclosed in U.S. Pat. No. 5,395,922 and European Patent No. 765,395A.

Likewise, the insulin precursor product for preparing the protracted, acylated insulin or insulin analogue to be mixed with the rapid acting single-chain insulin according to the invention can be produced by culturing a host cell containing a DNA sequence encoding the insulin precursor in question under conditions permitting the expression of the insulin precursor in question. The isolated insulin precursor can be acylated in the desired position as well know with the art and examples of such insulin analogues are described e.g. in the European patent applications having the publication Nos. EP 214826, EP 375437 and EP 383472.

The polynucleotide sequence coding for the respective insulin polypeptide may be pre-pared synthetically by established standard methods, e.g. the phosphoamidite method described by Beaucage et al. (1981) Tetrahedron Letters 22:1859-1869, or the method described by Matthes et al. (1984) EMBO Journal 3:801-805. According to the phosphoamidite method, oligonucleotides are synthesized, for example, in an automatic DNA synthesizer, purified, duplexed and ligated to form the synthetic DNA construct. A currently preferred way of preparing the DNA construct is by polymerase chain reaction (PCR).

The polynucleotide sequences may also be of mixed genomic, cDNA, and synthetic origin. For example, a genomic or cDNA sequence encoding a leader peptide may be joined to a genomic or cDNA sequence encoding the A and B chains, after which the DNA sequence may be modified at a site by inserting synthetic oligonucleotides encoding the desired amino acid sequence for homologous recombination in accordance with well-known procedures or preferably generating the desired sequence by PCR using suitable oligonucleotides.

The recombinant vector capable of replicating in the selected microorganism or host cell and which carries a polynucleotide sequence encoding the insulin polypeptide in question may be an autonomously replicating vector, i.e., a vector which exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used. The vector may be linear or closed circular plasmids and will preferably contain an element(s) that permits stable integration of the vector into the host cell's genome or autonomous replication of the vector in the cell independent of the genome.

In one embodiment, the recombinant expression vector is capable of replicating in yeast. Examples of sequences which enable the vector to replicate in yeast are the yeast plasmid 2 μm replication genes REP 1-3 and origin of replication.

The vectors may contain one or more selectable markers which permit easy selection of transformed cells. A selectable marker is a gene the product of which provides for biocide or viral resistance, resistance to heavy metals, prototrophy to auxotrophs, and the like. Examples of bacterial selectable markers are the dal genes from Bacillus subtilis or Bacillus licheniformis, or markers which confer antibiotic resistance such as ampicillin, kanamycin, chloramphenicol or tetracycline resistance. Selectable markers for use in a filamentous fungal host cell include amdS (acetamidase), argB (ornithine carbamoyltransferase), pyrG (orotidine-5′-phosphate decarboxylase) and trpC (anthranilate synthase. Suitable markers for yeast host cells are ADE2, HIS3, LEU2, LYS2, MET3, TRP1, and URA3. A well suited selectable marker for yeast is the Schizosaccharomyces pompe TPI gene (Russell (1985) Gene 40:125-130).

In the vector, the polynucleotide sequence is operably connected to a suitable promoter sequence. The promoter may be any nucleic acid sequence which shows transcriptional activity in the host cell of choice including mutant, truncated, and hybrid promoters, and may be obtained from genes encoding extra-cellular or intra-cellular polypeptides either homologous or heterologous to the host cell.

Examples of suitable promoters for directing the transcription in a bacterial host cell are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), and Bacillus licheniformis penicillinase gene (penP). Examples of suitable promoters for directing the transcription in a filamentous fungal host cell are promoters obtained from the genes for Aspergillus oryzae TAKA amylase, Rhizomucor miehei aspartic proteinase, Aspergillus niger neutral alpha-amylase, and Aspergillus niger acid stable alpha-amylase. In a yeast host, useful promoters are the Saccharomyces cerevisiae Ma1, TPI, ADH or PGK promoters.

The polynucleotide construct will also typically be operably connected to a suitable terminator. In yeast a suitable terminator is the TPI terminator (Alber et al. (1982) J. Mol. Appl. Genet. 1:419-434).

The procedures used to ligate the individual polynucleotide sequences contained in the expression vector such as DNA coding for the desired insulin polypeptide, the promoter and the terminator, respectively, and to insert them into a suitable vector containing the information necessary for replication in the selected host, are well known to persons skilled in the art. It will be understood that the vector may be constructed either by first preparing a DNA construct containing the entire DNA sequence encoding the single-chain insulins of the invention, and subsequently inserting this fragment into a suitable expression vector, or by sequentially inserting DNA fragments containing genetic information for the individual elements (such as the signal, pro-peptide, connecting peptide, A and B chains) followed by ligation.

The vector comprising such polynucleotide sequence is introduced into the host cell so that the vector is maintained as a chromosomal integrant or as a self-replicating extra-chromosomal vector as described earlier. The term “host cell” encompasses any progeny of a parent cell that is not identical to the parent cell due to mutations that occur during replication. The host cell may be a unicellular microorganism, e.g., a prokaryote, or a non-unicellular microorganism, e.g., a eukaryote. Useful unicellular cells are bacterial cells such as gram positive bacteria including, but not limited to, a Bacillus cell, Streptomyces cell, or gram negative bacteria such as E. coli and Pseudomonas sp. Eukaryote cells may be mammalian, insect, plant, or fungal cells. In a preferred embodiment, the host cell is a yeast cell. The yeast organism used in the process of the invention may be any suitable yeast organism which, on cultivation, produces large amounts of the single chain insulin of the invention. Examples of suitable yeast organisms are strains selected from the yeast species Saccharomyces cerevisiae, Saccharomyces kluyveri, Schizosaccharomyces pombe, Sacchoromyces uvarum, Kluyveromyces lactis, Hansenula polymorpha, Pichia pastoris, Pichia methanolica, Pichia kluyveri, Yarrowia lipolytica, Candida sp., Candida utilis, Candida cacaoi, Geotrichum sp., and Geotrichum fermentans.

The transformation of the yeast cells may for instance be effected by protoplast formation followed by transformation in a manner known per se. The medium used to cultivate the cells may be any conventional medium suitable for growing yeast organisms. The secreted insulin polypeptide, a significant proportion of which will be present in the medium in correctly processed form, may be recovered from the medium by conventional procedures including separating the yeast cells from the medium by centrifugation, filtration or catching the insulin precursor by an ion exchange matrix or by a reverse phase absorption matrix, precipitating the proteinaceous components of the supernatant or filtrate by means of a salt, e.g. ammonium sulphate, followed by purification by a variety of chromatographic procedures, e.g. ion exchange chromatography, affinity chromatography, or the like.

### Pharmaceutical Compositions

Compositions containing single-chain insulins of this invention optionally in mixture with a protracted acylated insulin analogue can be used in the treatment of states which are sensitive to insulin. Thus, they can be used in the treatment of type 1 diabetes, type 2 diabetes and hyperglycaemia for example as sometimes seen in seriously injured persons and persons who have undergone major surgery. The optimal dose level for any patient will depend on a variety of factors including the efficacy of the specific insulin derivative employed, the age, body weight, physical activity, and diet of the patient, on a possible combination with other drugs, and on the severity of the state to be treated. It is recommended that the daily dosage of the insulin derivative of this invention be determined for each individual patient by those skilled in the art in a similar way as for known insulin compositions.

Usually, the pharmaceutical composition of this invention is administered subcutaneously. However the composition may also be used in insulin pumps and may be formulated for pulmunal administration.

The pharmaceutical composition will contain usual adjuvants and additives and are preferably formulated as an aqueous solution. The aqueous medium is made isotonic, for example, with sodium chloride, sodium acetate or glycerol. Furthermore, the aqueous medium may contain zinc ions, buffers and preservatives. The pH value of the composition is adjusted to the desired value and may be between about 4 to about 8.5, preferably between 7 and 7.5 depending on the isoelectric point, pl, of the single-chain insulin in question.

The single-chain insulin analogue and the acylated long acting insulin can be mixed in a ratio of from about 10/90% about 30/70%, or about 50/50%.

In one embodiment the Pharmaceutical the molar ratio between the long acting, acylated insulin and the rapid acting, single chain insulin is greater than 2/1.

The buffer used in the pharmaceutical preparation according to the present invention may be selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

The pharmaceutically acceptable preservative may be selected from the group consisting of phenol, o-cresol, m-cresol, p-cresol, methyl p-hydroxybenzoate, propyl p-hydroxybenzoate, 2-phenoxyethanol, butyl p-hydroxybenzoate, 2-phenylethanol, benzyl alcohol, chlorobutanol, and thiomerosal, bronopol, benzoic acid, imidurea, chlorohexidine, sodium dehydroacetate, chlorocresol, ethyl p-hydroxybenzoate, benzethonium chloride, chlorphenesine (3p-chlorphenoxypropane-1,2-diol) or mixtures thereof. In a further embodiment of the invention the preservative is present in a concentration from 0.1 mg/ml to 20 mg/ml. In a further embodiment of the invention the preservative is present in a concentration from 0.1 mg/ml to 5 mg/ml. In a further embodiment of the invention the preservative is present in a concentration from 5 mg/ml to 10 mg/ml. In a further embodiment of the invention the preservative is present in a concentration from 10 mg/ml to 20 mg/ml. Each one of these specific preservatives constitutes an alternative embodiment of the invention. The use of a preservative in pharmaceutical compositions is well-known to the skilled person. For convenience reference is made to Remington: The Science and Practice of Pharmacy, 19th edition, 1995.

The isotonicity agent may be selected from the group consisting of a salt (e.g. sodium chloride), a sugar or sugar alcohol, an amino acid (e.g. L-glycine, L-histidine, arginine, lysine, isoleucine, aspartic acid, tryptophan, threonine), an alditol (e.g. glycerol (glycerine), 1,2-propanediol (propyleneglycol), 1,3-propanediol, 1,3-butanediol) polyethyleneglycol (e.g. PEG400), or mixtures thereof. Any sugar such as mono-, di-, or polysaccharides, or water-soluble glucans, including for example fructose, glucose, mannose, sorbose, xylose, maltose, lactose, sucrose, trehalose, dextran, pullulan, dextrin, cyclodextrin, soluble starch, hydroxyethyl starch and carboxymethylcellulose-Na may be used. In one embodiment the sugar additive is sucrose. Sugar alcohol is defined as a C4-C8 hydrocarbon having at least one —OH group and includes, for example, mannitol, sorbitol, inositol, galactitol, dulcitol, xylitol, and arabitol. In one embodiment the sugar alcohol additive is mannitol. The sugars or sugar alcohols mentioned above may be used individually or in combination. There is no fixed limit to the amount used, as long as the sugar or sugar alcohol is soluble in the liquid preparation and does not adversely effect the stabilizing effects achieved using the methods of the invention. In one embodiment, the sugar or sugar alcohol concentration is between about 1 mg/ml and about 150 mg/ml. In a further embodiment of the invention the isotonic agent is present in a concentration from 1 mg/ml to 50 mg/ml. In a further embodiment of the invention the isotonic agent is present in a concentration from 1 mg/ml to 7 mg/ml. In a further embodiment of the invention the isotonic agent is present in a concentration from 8 mg/ml to 24 mg/ml. In a further embodiment of the invention the isotonic agent is present in a concentration from 25 mg/ml to 50 mg/ml. Each one of these specific isotonic agents constitutes an alternative embodiment of the invention. The use of an isotonic agent in pharmaceutical compositions is well-known to the skilled person. For convenience reference is made to Remington: The Science and Practice of Pharmacy, 19th edition, 1995.

By a single-chain insulin is meant a polypeptide sequence of the general structure B-C-A wherein B is the human B insulin chain or an analogue or derivative thereof, A is the human insulin A chain or an analogue or derivative and C is a peptide chain of 6-10 amino acid residues connecting the C-terminal amino acid residue in the B-chain (normally B30) with A1. If the B chain is a desB30 chain the connecting peptide will connect B29 with A1, The single-chain insulin will contain correctly positioned disulphide bridges (three) as in human insulin that is between CysA7 and CysB7 and between CysA20 and CysB19 and an internal disulfide bridge between CysA6 and CysA11.

With “SCI” is meant single-chain insulin.

Analogues of the B-chain may be such wherein the amino acid residue in B1 is substituted with another amino acid residue such as Asp or Gly or is deleted. Also Asn at position B3 may be mutated with Thr, Lys, Gln, Glu or Asp. Further examples of analogues of the B-chain are such wherein the B30 amino acid residue is missing. The B-chain may also comprise an N-terminal extension.

Analogues of the A chain may be such wherein the amino acid residue in position A18 is substituted with another amino acid residue, such as Gln. Also, Asn at position A21 may be mutated with Ala, Gln, Glu, Gly, His, Ile, Leu, Met, Ser, Thr, Trp, Tyr or Val, in particular with Gly, Ala, Ser, or Thr and preferably with Gly. Finally the A-chain may comprise a C-terminal extension.

With desB30 or B(1-29) is meant a natural insulin B chain or an analogue thereof lacking the B30 amino acid residue, A(1-21) means the natural insulin A chain or an analogue or derivative thereof. The amino acid residues are indicated in the three letter amino acid code or the one letter amino code.

With B1, A1 etc. is meant the amino acid residue in position 1 in the B chain of insulin (counted from the N-terminal end) and the amino acid residue in position 1 in the A chain of insulin (counted from the N-terminal end), respectively.

The single-chain insulin analogues are primarily named according to the following rule: The sequence starts with the B-chain, continues with the C-peptide and ends with the A-chain. The amino acid residues are named after their respective counterparts in human insulin and mutations are explicitly described whereas unaltered amino acid residues in the A- and B-chains are not mentioned. For example, an insulin having the following mutations as compared to human insulin A21Gly, B3Gln, B10Gln, B28Glu, A18Gln and desB30 and comprising the C-peptide TGLGSGK (SEQ ID NO:3) connecting the C-terminal B-chain and the N-terminal A-chain is named B(1-29)-B3Gln-B10Gln-B28Glu-TGLGSGK (SEQ ID NO:3)-A(1-21)-A18Gln-A21 Gly human insulin.

The term “monomeric insulin”, when used herein, refers to human insulin analogs that are less prone to self-association (into dimers and hexamers) than human insulin.

With fast acting insulin is meant insulin having a faster onset of action than normal or regular human insulin.

With long acting insulin is meant insulin having a longer duration of action than normal or regular human insulin.

A polypeptide with Insulin receptor and IGF-1 receptor affinity is a polypeptide which is capable of interacting with an insulin receptor and an human IGF-1 receptor in a suitable binding assay.

POT” is the Schizosaccharomyces pombe triose phosphate isomerase gene, and “TPI1” is the S. cerevisiae triose phosphate isomerase gene.

By a “leader” is meant an amino acid sequence consisting of a pre-peptide (the signal peptide) and a pro-peptide.

The term “signal peptide” is understood to mean a pre-peptide which is present as an N-terminal sequence on the precursor form of a protein. The function of the signal peptide is to allow the heterologous protein to facilitate translocation into the endoplasmic reticulum. The signal peptide is normally cleaved off in the course of this process. The signal peptide may be heterologous or homologous to the yeast organism producing the protein. A number of signal peptides which may be used with the DNA construct of the invention including yeast aspartic protease 3 (YAP3) signal peptide or any functional analog (Egel-Mitani et al. (1990) YEAST 6:127-137 and U.S. Pat. No. 5,726,038) and the α-factor signal of the MFα1 gene (Thorner (1981) in The Molecular Biology of the Yeast Saccharomyces cerevisiae, Strathern et al., eds., pp 143-180, Cold Spring Harbor Laboratory, NY and US 4,870,00.

The term “pro-peptide” means a polypeptide sequence whose function is to allow the expressed polypeptide to be directed from the endoplasmic reticulum to the Golgi apparatus and further to a secretory vesicle for secretion into the culture medium (i.e. exportation of the polypeptide across the cell wall or at least through the cellular membrane into the periplasmic space of the yeast cell). The pro-peptide may be the yeast α-factor pro-peptide, vide U.S. Pat. Nos. 4,546,082 and 4,870,008. Alternatively, the pro-peptide may be a synthetic pro-peptide, which is to say a pro-peptide not found in nature. Suitable synthetic pro-peptides are those disclosed in U.S. Pat. Nos. 5,395,922; 5,795,746; 5,162,498 and WO 98/32867. The pro-peptide will preferably contain an endopeptidase processing site at the C-terminal end, such as a Lys-Arg sequence or any functional analogue thereof.

In the present context the three-letter or one-letter indications of the amino acids have been used in their conventional meaning as indicated in the following table. Unless indicated explicitly, the amino acids mentioned herein are L-amino acids. Further, the left and right ends of an amino acid sequence of a peptide are, respectively, the N- and C-termini unless otherwise specified.

Abbreviations for amino acids

| | | One-letter |
|---|---|---|
| Amino acid | Tree-letter code | code |
| Glycine | Gly | G |
| Proline | Pro | P |
| Alanine | Ala | A |
| Valine | Val | V |
| Leucine | Leu | L |
| Isoleucine | Ile | I |
| Methionine | Met | M |
| Cysteine | Cys | C |
| Phenylalanine | Phe | F |
| Tyrosine | Tyr | Y |
| Tryptophan | Trp | W |
| Histidine | His | H |
| Lysine | Lys | K |
| Arginine | Arg | R |
| Glutamine | Gln | Q |
| Asparagine | Asn | N |
| Glutamic Acid | Glu | E |
| Aspartic Acid | Asp | D |
| Serine | Ser | S |
| Threonine | Thr | T |

The present invention is described in further detail in the following examples which are not in any way intended to limit the scope of the invention as claimed. The attached Figure is meant to be considered as integral parts of the specification and description of the invention. All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

### General Procedures

All expressions plasmids are of the C-POT type, similar to those described in EP 171, 142, which are characterized by containing the Schizosaccharomyces pombe triose phosphate isomerase gene (POT) for the purpose of plasmid selection and stabilization in S. cerevisiae. The plasmids also contain the S. cerevisiae triose phosphate isomerase promoter and terminator. These sequences are similar to the corresponding sequences in plasmid pKFN1003 (described in WO 90/100075) as are all sequences except the sequence of the EcoRI-XbaI fragment encoding the fusion protein of the leader and the insulin product. In order to express different fusion proteins, the EcoRI-XbaI fragment of pKFN1003 is simply replaced by an EcoRI-XbaI fragment encoding the leader-insulin fusion of interest. Such EcoRI-XbaI fragments may be synthesized using synthetic oligonucleotides and PCR according to standard techniques.

Yeast transformants were prepared by transformation of the host strain S. cerevisiae strain MT663 (MA Ta/MATα pep4-3/pep4-3 HIS4/his4 tpi::LEU21tpi::LEU2 Cir⁺). The yeast strain MT663 was deposited in the Deutsche Sammlung von Mikroorganismen und Zellkulturen in connection with filing WO 92/11378 and was given the deposit number DSM 6278.

MT663 was grown on YPGaL (1% Bacto yeast extract, 2% Bacto peptone, 2% galactose, 1% lactate) to an O.D. at 600 nm of 0.6. 100 ml of culture was harvested by centrifugation, washed with 10 ml of water, recentrifuged and resuspended in 10 ml of a solution containing 1.2 M sorbitol, 25 mM Na₂EDTA pH=8.0 and 6.7 mg/ml dithiotreitol. The suspension was incubated at 30° C. for 15 minutes, centrifuged and the cells resuspended in 10 ml of a solution containing 1.2 M sorbitol, 10 mM Na₂EDTA, 0.1 M sodium citrate, pH 0 5.8, and 2 mg Novozym®234. The suspension was incubated at 30° C. for 30 minutes, the cells collected by centrifugation, washed in 10 ml of 1.2 M sorbitol and 10 ml of CAS (1.2 M sorbitol, 10 mM CaCl₂, 10 mM Tris HCl (Tris=Tris(hydroxymethyl)aminomethane) pH=7.5) and resuspended in 2 ml of CAS. For transformation, 1 ml of CAS-suspended cells was mixed with approx. 0.1 mg of plasmid DNA and left at room temperature for 15 minutes. 1 ml of (20% polyethylene glycol 4000, 10 mM CaCl₂, 10 mM Tris HCl, pH=7.5) was added and the mixture left for a further 30 minutes at room temperature. The mixture was centrifuged and the pellet resuspended in 0.1 ml of SOS (1.2 M sorbitol, 33% v/v YPD, 6.7 mM CaCl₂) and incubated at 30° C. for 2 hours. The suspension was then centrifuged and the pellet resuspended in 0.5 ml of 1.2 M sorbitol. Then, 6 ml of top agar (the SC medium of Sherman et al. (1982) Methods in Yeast Genetics, Cold Spring Harbor Laboratory) containing 1.2 M sorbitol plus 2.5% agar) at 52° C. was added and the suspension poured on top of plates containing the same agar-solidified, sorbitol containing medium.

S. cerevisiae strain MT663 transformed with expression plasmids was grown in YPD for 72 h at 30° C.

### Example 1

The following fast acting insulin analogues were tested:

B(1-29)-B10Gln-B28Glu-TGLGSGK (SEQ ID NO:)-A(1-21)-A18Gln human insulin (compound A) and B(1-29)-B1Gly-B3Gln-B10Gln-B28Glu-TGLGSGK (SEQ ID NO:)-A(1-21)-A18Gln-A21Gly human insulin (compound B).

The fast acting single-chain analogues were tested alone or in combinations with the following prolonged acting insulins: Lys^{B29} (N^{ε}-tetradecanoyl) des(B30) human insulin (insulin detemir) (compound C), Lys^{B29} (N^{ε}—(N-lithocholyl-γ-glutamyl)) des(B30) human insulin (compound D) and N^{εB29}—(N^{α}—(HOOC(CH₂)₁₄CO)-γ-Glu) des(B30) human insulin (compound E).

Migration of the insulin analogues and insulin analogue combinations were measured in relation to the standards mentioned below. The theoretical migration pattern for each sample combination has been calculated from the migration pattern of each of the analogues alone.

The test mixtures were formulated with conventional pharmaceutical adjuvants and additives. All mixtures were formulated with 16/16 mM phenol/cresol. Zinc was added as zinc acetate in various amounts. pH was 7.5. The rapid acting SCI analogues are mixable with the long acting insulin when they do not differ more than 15% on any of the components from the theoretical value.

The results appear from the following table.

TABLE 1

| | | | | | | Theoretical | |
|---|---|---|---|---|---|---|---|
| | | | | | | SEC | |
| Com- | Com- | Com- | Com- | Com- | | Migration | Measured SEC |
| pound A | pound B | pound C | pound D | pound E | Zn | % X2/% HSA | migration |
| mM | mM | mM | mM | mM | mM | or aggregate | % X2/% HSA |
| 0.6 | | | | | | 100/0 | 100/0 |
| | 0.6 | | | | | 100/0 | 100/0 |
| | | 2.4 | | | 1.4 | | 25/75 |
| | | | 1.2 | | 0.6 | | 15/78 |
| | | | | 1 | 0.5 | | 13/78 |
| 0.3 | | 0.3 | | | 0.3 | 63/37 | 88/11 |
| 0.3 | | 0.6 | | | 0.45 | 50/50 | 64/35 |
| 0.2 | | 0.8 | | | 0.5 | 40/60 | 49/51 |
| 0.2 | | 1.7 | | | 1 | 80/20 | 68/32 |
| 0.6 | | 2.4 | | | 1.2 | 38/62 | 33/67 |
| 0.6 | | 2.4 | | | 1.3 | 38/62 | 37/62 |
| 0.6 | | 2.4 | | | 1.4 | 38/62 | 37/63 |
| 0.6 | | 2.4 | | | 1.5 | 38/62 | 39/61 |
| 0.6 | | 2.4 | | | 1.6 | 38/62 | 43/55 |
| 0.6 | | 2.4 | | | 1.8 | 38/62 | 46/54 |
| 0.6 | | 2.4 | | | 2.0 | 38/62 | 44/56 |
| 0.6 | | | 1.2 | | 0.6 | 43/57 | 37/59 |
| 0.6 | | | 1.2 | | 0.65 | 43/57 | 42/56 |
| 0.6 | | | 1.2 | | 0.7 | 43/57 | 42/56 |
| 0.6 | | | 1.2 | | 0.75 | 43/57 | 41/58 |
| 0.6 | | | 1.2 | | 0.8 | 43/57 | 40/57 |
| 0.6 | | | 1.2 | | 0.9 | 43/57 | 38/58 |
| 0.6 | | | 1.2 | | 1.0 | 43/57 | 43/53 |
| 0.3 | | | 0.3 | | 0.3 | 58/42 | 66/25 |
| 0.3 | | | 0.6 | | 0.45 | 43/57 | 43/53 |
| 0.2 | | | 0.8 | | 0.5 | 32/62 | 28/67 |
| 0.3 | | | | 0.3 | 0.3 | 56/39 | 54/41 |
| 0.3 | | | | 0.6 | 0.45 | 41/52 | 35/64 |
| 0.2 | | | | 0.8 | 0.5 | 30/63 | 33/59 |
| 0.6 | | | | 1 | 0.8 | 45/49 | 37/59 |
| | 0.3 | 0.3 | | | 0.3 | 63/37 | 100/0 |
| | 0.3 | 0.6 | | | 0.45 | 50/50 | 46/50 |
| | 0.2 | 0.8 | | | 0.5 | 40/60 | 49/51 |
| | 0.6 | 2.4 | | | 1.2 | 38/62 | 49/51 |
| | 0.6 | 2.4 | | | 1.3 | 38/62 | 39/61 |
| | 0.6 | 2.4 | | | 1.4 | 38/62 | 39/61 |
| | 0.6 | 2.4 | | | 1.5 | 38/62 | 36/62 |
| | 0.6 | 2.4 | | | 1.6 | 38/62 | 38/62 |
| | 0.6 | 2.4 | | | 1.8 | 38/62 | 33/67 |
| | 0.6 | 2.4 | | | 2.0 | 38/62 | 33/67 |
| | 0.3 | | 0.3 | | 0.3 | 58/42 | 62/34 |
| | 0.3 | | 0.6 | | 0.45 | 43/57 | 42/58 |
| | 0.2 | | 0.8 | | 0.5 | 32/62 | 27/73 |
| | 0.6 | | 1.2 | | 0.6 | 43/57 | 44/51 |
| | 0.6 | | 1.2 | | 0.7 | 43/57 | 44/51 |
| | 0.6 | | 1.2 | | 0.8 | 43/57 | 43/52 |
| | 0.6 | | 1.2 | | 0.9 | 43/57 | 41/54 |
| | 0.6 | | 1.2 | | 1.0 | 43/57 | 40/55 |
| | 0.3 | | | 0.3 | 0.3 | 56/39 | 57/35 |
| | 0.3 | | | 0.6 | 0.45 | 41/52 | 54/40 |
| | 0.2 | | | 0.8 | 0.5 | 30/63 | 33/58 |
| | 0.6 | | | 1 | 0.8 | 45/49 | 42/54 |

### Pharmacological Methods

### Biochemical Methods

### Assay (1)

Size exclusion chromatography (SEC) was performed essentially as described in Havelund et al. 2004 Pharmaceutical research, 21, 1498-1504. The chromatographic system was a Superose 6 HR PC 6/30 column (GE healthcare) eluted by tris-buffered isotonic saline (NaCl 140 mM, tris/HCl 10 mM, NaN3 0.01%, pH 7.5 at 37° C., injecting 1% of column volume and using a flow of 90 min per column volume and UV detection at 276 nm. The references were a stable insulin monomeric insulin X2 (AspB9, GluB27 human insulin, zinc free), a stable hexameric insulin: Co(III) insulin, albumin (HSA), and covalent albumin (formed in solution).

### Assay (II)

Insulin Receptor Binding of the Single-Chain Insulin

The affinity of the single-chain insulins for the human insulin receptor can be determined by a SPA assay (Scintillation Proximity Assay) microtiterplate antibody capture assay. SPA-PVT antibody-binding beads, anti-mouse reagent (Amersham Biosciences, Cat No. PRNQ0017) are mixed with 25 ml of binding buffer (100 mM HEPES pH 7.8; 100 mM sodium chloride, 10 mM MgSO4, 0.025% Tween-20). Reagent mix for a single Packard Optiplate (Packard No. 6005190) is composed of 2.4 μl of a 1:5000 diluted purified recombinant human insulin receptor-exon 11, an amount of a stock solution of A14 Tyr[125I]-human insulin corresponding to 5000 cpm per 100 μl of reagent mix, 12 μl of a 1:1000 dilution of F12 antibody, 3 ml of SPA-beads and binding buffer to a total of 12 ml. A total of 100 μl is then added and a dilution series is made from appropriate samples. To the dilution series is then added 100 μl of reagent mix and the samples are incubated for 16 hours while gently shaken. The phases are then separated by centrifugation for 1 min and the plates counted in a Topcounter. The binding data are fitted using the nonlinear regression algorithm in the GraphPad Prism 2.01 (GraphPad Software, San Diego, Calif.).

### Assay (III)

Alternatively the insulin receptor binding may be tested in a hIRBHK membran assay as follows:

Binding of [¹²⁵I]-human insulin to membrane-associated recombinant human insulin receptor isoform A (hIR-A)

### Reagents:

¹²⁵I-Insulin: Novo Nordisk A/S, mono ¹²⁵I-(TyrA14) human insulin

### Human Insulin: Novo Nordisk A/S,

Human serum albumin: Dade Behring, ORHA 194 C30, lot 455077
Plastic ware: Packard OptiPlate™-96, #6,005,290
Scintillant: Amersham Biosciences, WGA coated PVT microspheres, # RPNQ0001
Cells: BHK tk⁻ ts13 cells expressing recombinant human insulin receptor isoform A (hIR12-14).

Extraction of membrane-associated insulin receptors: BHK cells from a ten-layer cell factory are harvested and homogenised in 25 ml of ice-cold buffer (25 mM HEPES pH 7.4, 2.5 mM CaCl₂, 1 mM MgCl₂, 250 mg/l bacitracin, 0.1 mM Pefablock). The homogenate is layered carefully on 41% sucrose cushions, centrifuged in the ultracentrifuge at 95,000×g for 75 minutes in a Beckman SW28 rotor at 4° C. The plasma membranes are collected from the top of the sucrose cushion, diluted 1:4 with buffer and centrifuged at 40,000×g for 45 min in a Beckman SW28 rotor. The pellets are suspended in buffer (25 mM HEPES pH 7.4, 2.5 mM CaCl₂, 1 mM MgCl₂, 250 mg/l bacitracin, 0.1 mM Pefablock) and stored at −80° C.

Radioligand binding to membrane-associated insulin receptors is performed in duplicate in 96-well OptiPlates. Membrane protein is incubated for 150 minutes at 25° C. with 50 pM [125I-TyrA14]-human insulin in a total volume of 200 ml assay buffer (50 mM HEPES, 150 mM NaCl, 5 mM MgSO4, 0.01% Triton X-100, 0.1% HSA, Complete™ EDTA-free protease inhibitors) and increasing concentrations of human insulin or insulin analogues (typically between 0.01 and 300 nM). The assay is terminated by addition of 50 μl of a suspension of WGA-coated PVT microspheres (20 mg/ml). Following 5 minutes of slight agitation, the plate is centrifuged at 1500 RPM for 6 minutes, and bound radioactivity quantified by counting in a Packard TopCount NXT after a delay of 60 minutes.

Results are given as IC₅₀ relative to human insulin in %.

### Assay (IV)

Potency of the Single-Chain Insulin Analogues Relative to Human Insulin.

Wistar rats are used for testing the blood glucose lower efficacy of SCI af I.V bolus administration. Following administration of either SCI or human insulin the concentration of blood glucose is monitored.

1. A single-chain insulin analogue comprising (i) the A-chain of human insulin or an analogue thereof and (ii) a modified B-chain, wherein (i) and (ii) are connected by a connecting peptide and wherein
a) one or more of the amino acid residues in position B25, B26 or B27 in the human B-chain are Glu or Asp or are deleted and/or the amino acid residue in position B28 is Glu, Asp, Lys or is deleted,
b) the amino acid residue in position B10 in the human insulin B-chain is selected from the group consisting of Gln, Ale, Val, Thr and Ser; and
c) optionally the amino acid residues in position B22 is Glu or Asp
provided that if B28 is Lys then B29 is Pro.

2. An insulin analogue according to claim 1, wherein the amino acid residues in position B22, B25, B26 and B27 are the natural amino acid at that position in the human B-chain and the amino acid residue in position B28 is Glu or Asp.

3. An insulin analogue according to claim 1, wherein the amino acid residue in position B10 in the human insulin B-chain is Gln.

4. An insulin analogue according to claim 1, wherein the connecting peptide has from 6-10 amino acid residues in the chain.

5. An insulin analogue according to claim 1, wherein the amino acid residue in position A21 is Gly.

6. An insulin analogue according to claim 1, wherein the amino acid residue in position B1 is Gly or is deleted.

7. An insulin analogue according to claim 1, wherein the amino acid residue in position B3 is Gln.

8. An insulin analogue according to claim 1, wherein the amino acid residue in position B30 is deleted.

9. A pharmaceutical composition comprising an insulin analogue according to claim 1 and a long acting, acylated insulin analogue.

10. A pharmaceutical composition according to claim 9, wherein the long acting, acylated insulin is Lys^{B29}(N^{ε}-tetradecanoyl) des(B30) human insulin (insulin detemir).
